(19) United States
(12) Patent Application Publication
(10) Pub. No.: US 2025/0345315 A1
(43) Pub. Date: Nov. 13, 2025

(54) Dispenseable formulation for active pharmaceutical ingredients

(71) Applicant: CurifyLabs Oy, Helsinki (FI)

(72) Inventors: Niklas Sandler, Helsinki (FI); Sari Airaksinen, Helsinki (FI); Soumya Verma, Helsinki (FI)

(21) Appl. No.: 18/660,279

(22) Filed: May 10, 2024

(51) Int. Cl. A61K 31/4365; A61K 9/20; A61K 31/138; A61K 31/357

(52) U.S. Cl. CPC A61K 31/4365; A61K 9/2009; A61K 9/2013; A61K 9/2018; A61K 9/2063; A61K 9/2068; A61K 9/2095; A61K 31/138; A61K 31/357

(57) ABSTRACT

The present disclosure relates to formulations which can be admixed with active pharmaceutical ingredients (API) to produce dispenseable dispersions. The disclosure also relates to methods for preparing solid dosage formulations of APIs using the formulations. The formulations include 5-30 wt.-% one or more gelling agents, 0.5-15 wt.-% silica, 10-35 wt.-% of one or more lipid-based excipients, such as cocoa butter and/or hydrated coconut oil, 10-30 wt.-% carbohydrates and/or sugar alcohols, and 40-70 wt.-% one or more solvents.

### FIELD

The present disclosure relates to formulations which can be admixed with active pharmaceutical ingredients (API) to produce dispenseable dispersions. The disclosure also relates to methods for preparing solid dosage formulations of APIs using the formulations.

### BACKGROUND

Extrusion-based dispensing is a technology which gives the possibility to manufacture personalized dosage forms of medicaments. The main benefit of the technology for personalized pharmaceuticals is the ability to produce small batches with carefully tailored dosages, shapes, sizes, and release characteristics. It also allows flavors to be incorporated into a dose without the need of a film coating, entirely masking the taste of chemical compounds. Dispensers can be installed in pharmacies, hospitals, clinics, and remote locations, enabling on-demand production of drugs, particularly those with poor stability or that have cold chain storage requirements. For pharmaceutical companies and pharmacists, extrusion- based dispensing can significantly reduce costs, waste, and environmental burden as dispensers only deposit the exact amounts of raw materials required.

The main challenge of the extrusion-based dispensing process is the development of the dispenseable formulation itself because its physiochemical properties could strongly affect the dispenseability. The formulation should be dispenseable at moderate temperature while solid at ambient temperature. Furthermore, the formulation should be suitable for a broad range of active pharmaceutical ingredients.

Accordingly, there is still a need for further dispenseable formulations for active pharmaceutical ingredients

### SUMMARY

In one aspect, it is an object of the present invention to provide a dispenseable formulation for active pharmaceutical ingredients, the formulation comprising:
5-30 wt.-% of one or more gelling agents selected from a group consisting of gelatin, agar, and combinations thereof;
0.5-5 wt.-% silica;
10-35 wt.-% one or more lipid-based excipients selected from one or more hydrated vegetable fats, wherein the melting point of the one or more hydrated vegetable fats is from 35° C. to 55° C.;
10-30 wt.-% of one or more carbohydrates and/or sugar alcohols; and
40-70 wt.-% one or more solvents.

It is also an object of the present disclosure to provide a dispersion including one or more active pharmaceutical ingredients and a formulation comprising:
5-30 wt.-% of one or more gelling agents selected from a group consisting of gelatin, agar, and combinations thereof;
0.5-5 wt.-% silica;
10-35 wt.-% of one or more lipid-based excipients selected from one or more hydrated vegetable fats, wherein the melting point of the one or more hydrated vegetable fats is from 35° C. to 55° C.;
10-30 wt.-% of one or more carbohydrates and/or sugar alcohols; and
40-70 wt.-% of one or more solvents.

It is also an object of the present disclosure to provide a method for producing a solid dosage form of one or more active pharmaceutical ingredients, wherein the solid dosage form includes a formulation comprising:
5-30 wt.-% of one or more gelling agents selected from a group consisting of gelatin, agar, and combinations thereof;
0.5-5 wt.-% silica;
10-35 wt.-% of one or more lipid-based excipients selected from one or more hydrated vegetable fats, wherein the melting point of the one or more hydrated vegetable fats is from 35° C. to 55° C.;
10-30 wt.-% of one or more carbohydrates and/or sugar alcohols; and
40-70 wt.-% of one or more solvents.

A number of exemplifying and non-limiting embodiments of the invention are described in accompanied dependent claims. Various exemplifying and non-limiting embodiments of the invention and methods of operation, together with additional objects and advantages thereof, are best understood from the following description of specific exemplifying embodiments, when read in connection with the accompanying figures.

The verbs “to comprise” and “to include” are used in this document as open limitations that neither exclude nor require the existence of further unrecited features. The features recited in depending claims are mutually freely combinable unless otherwise explicitly stated. Furthermore, it is to be understood that the use of “a” or “an”, i.e., a singular form, throughout this document does not exclude a plurality.

### DESCRIPTION

The specific examples provided in the description given below should not be construed as limiting the scope and/or the applicability of the appended claims. Lists and groups of examples provided in the description given below are not exhaustive unless otherwise explicitly stated.

According to one aspect the present disclosure concerns a dispenseable formulation comprising:
5-30 wt.-% of one or more gelling agents selected from a group consisting of gelatin, agar, and combinations thereof;
0.5-5 wt.-% silica;
10-35 wt.-% of one or more lipid-based excipients selected from one or more hydrated vegetable fats, wherein the melting point of the one or more hydrated vegetable fats is from 35° C. to 55° C.;
10-30 wt.-% of one or more carbohydrates and/or sugar alcohols;
40-70 wt.-% of one or more solvents.

In one embodiment, the gelling agent comprises gelatin. Gelatin, due to its thermo reversibility, may be favorable when there is a need for reheating and melting the formulation. According to one embodiment, the formulation comprises 10-15 wt.-% gelatin.

According to another embodiment, the gelling agent is agar. Agar is generally not as good a gelling agent as gelatin since its gelation temperature is ca 80° C. Although agar as such does not possess thermosensitive properties, it can be grafted, e.g., with polyvinyl caprolactam, to give rise to a thermoresponsive hydrogel. According to an embodiment, the formulation includes 5-10% agar, such as 6 wt.-% agar.

The formulation must have certain viscosity so that the API does not separate from the formulation, which, in some embodiments, comprises at least 40 wt.-% solvent, such as water. This amount may be of high importance in storage and during dispensing processes. In some embodiments, the viscosity is adjusted by silica (silicon dioxide).

The amount of a lipid-based excipient in the formulation is from 10-35 wt.-% for more accurate control of viscoelastic behavior and ease of processing. If the amount is lower, the consistency is less favorable due to low viscosities in optimal processing temperatures. With greater wt.-%'s, separation of the oil and water phase is more prone to occur. Also, higher temperatures may be needed due to increased viscosity. Furthermore, too great of a wt.-% of a lipid-based excipient in the formulation may result in a fat-like consistency and its taste may be unpleasant. Exemplary suitable lipid-based excipients for use in the products and processes described herein are cocoa butter and hydrated coconut oil. Also, mixtures of cocoa butter and hydrated coconut oil can be used.

The formulation comprises 10-30 wt.-% carbohydrates and/or sugar alcohols. Exemplary carbohydrates are glucose, fructose, lactose, starch, maltodextrin, and cellulose. A particular sugar alcohol is xylitol, which may be used as a filler and a sweetener. According to a particular embodiment, the formulation includes 15-25 wt.-% xylitol, such as 20 wt.-% xylitol.

The formulation further comprises 40-70 wt.-% of one or more solvents. In an embodiment, the one or more solvents include water and/or glycerol. According to a particular embodiment, the formulation comprises 40-50 wt.-% water and 5-6 wt.-% glycerol. Higher solvent amounts, such as 50 wt.-% or more, may be preferable when there is a need to achieve high solubilization of the API in the formulation. However, the presence of too high solvent levels, especially water levels, but also one or more of further Near Infra-Red (NIR) absorbing elements, such as thickeners, further solvents, and fillers, should be avoided if NIR is used for quality control of the formulation.

In some embodiments, the formulation also includes one or more excipients selected from a group consisting of preservatives, pH-adjusting agents, chelating agents, sweeteners, surfactants, and flavoring agents. Chelating agents, such as EDTA, are known to increase stability of certain drugs, such as captopril. Surfactants, in turn, assist in stabilizing oil-in water emulsions by reducing the interfacial tension between oil and water phases, and by forming a protective layer around oil droplets to prevent them from coalescing. Exemplary surfactants include polysorbates.

Exemplary pH adjusting agents include citric acid, sodium citrate, and malic acid. Exemplary preservatives include sorbic acid, potassium sorbate, and parabens. Exemplary flavoring agents include marshmallow flavour, molasses flavor, vanilla flavour, peanut butter flavour, fruit flavour, and berry flavour. Exemplary sweeteners include the above-mentioned glucose, fructose, and xylitol. Also, artificial sweeteners, such as sucralose, and polyol sugars, such as erythritol which are not metabolized in the same way as normal carbohydrates, can be used.

According to a particular embodiment, the formulation includes 10-15 wt.-% gelatin as a gelling agent, 1-2 wt. % silicon dioxide as a thickening agent, 10-15 wt.-% of cocoa butter as a lipid-based excipient, and 20 wt.-% xylitol as a filler and a sweetener. A particular formulation is disclosed in Table 1.

TABLE 1

| Ingredient | wt.-% | |
|---|---|---|
| Gelatin | 12.42 | gelling agent |
| Water | 42.67 | solvent |
| Glycerol | 5.59 | solvent |
| Potassium sorbate | 0.15 | preservative |
| Citric acid | 0.47 | pH adjusting agent |
| Sodium citrate | 0.2 | pH adjusting agent |
| Sucralose | 0.048 | sweetener |
| Xylitol | 20.38 | sweetener and filler |
| Maltodextrin | 4.23 | filler |
| Cocoa butter | 12.23 | lipid-based excipient |
| Silicon dioxide | 1.23 | thickener |
| Raspberry flavor | 0.15 | flavor |
| Vanilla flavor | 0.23 | flavor |
| Total | 100.00 | |

It is essential that the formulation is thermoreversible. Its viscosity at the dispensing temperature must enable dispensing at moderate temperature, such as at 40-50° C., while it must be solid at room temperature. In some embodiments, the viscosity of the formulation at 50° C. (shear rate 150-800) is from 1-4 Pa·s. For example, the viscosity of the formulation of Table 1 is ca 2.1 Pa·s at 50° C.

The formulation of the present disclosure is suitable for dispensing a wide range of active pharmaceutical ingredients (API), independent of the properties of the API. Accordingly, dispenseable dispersions could be achieved when the formulation was admixed with 0.1-5 wt.-% one or more Class I, Class II, Class III, and/or Class IV APIs, wherein the wt.-% is the total weight of the APIs in the dispersion. When the one or more APIs were of Class I or III, the wt.-% range may be broader, i.e., 0.1-25 wt.-%. For example, a dispersion including 25 wt.-% propranolol and 75 wt.-% of a formulation as disclosed herein could be used for producing a solid dosage formulation of the API, whereas in the case of ramipril and quetiapine, the maximum amount of the API was 5 wt.-%.

Exemplary non-limiting APIs suitable for the present disclosure are disclosed in Table 2.

TABLE 2

| API | Class^{a} |
|---|---|
| Amlodipine besilate | I |
| Acetylsalicylic acid | I |
| Bisoprolol fumarate | I |
| Candesartan | II |
| Atenolol | III |
| Caffeine | I |
| Caffeine | I |
| Captopril | I |
| Clopidogrel bisulphate | II |
| Diltiazem HCl | I |
| Furosemide | IV |
| HCT (hydrochlorothiazide) | IV |
| Hydrocortisone | II |
| Ibuprofen | II |
| Melatonin | II |
| Metoprolol tartrate | I |
| Ondansetron HCl dihydrate | II |
| Paracetamol | I |
| Prednisolone | I |
| Propranolol HCl | I |
| Quetiapine fumarate | II |
| Ramipril | II |
| Sildenafil citrate | II |
| Spironolactone | II |
| Atenolol | III |
| Diltiazem | I |
| Gabapentin | III |
| Sotalol | I |
| Tacrolimus | II |
| Topiramate | II |
| Doxycycline | I |
| Ursodeoxycholic acid | II |
| Neomycin | III |
| Isoniazid | III |
| Nitrofurantoin | II |

^{a}Class I: high permeability, high solubility; Class II: high permeability, low solubility; Class III: low permeability, high solubility; Class IV: low permeability, low solubility (M9 Guideline Step4 2019 1116.pdf (ich.org)).

According to another aspect, the present disclosure concerns a method for producing a solid dosage formulation.

According to one embodiment the method comprising the steps of:
a) providing a formulation comprising:
   5-30 wt.-% of one or more gelling agents selected from a group consisting of gelatin, agar, and combinations thereof;
   0.5-5 wt.-% silica;
   10-35 wt.-% of one or more lipid-based excipients selected from one or more hydrated vegetable fats, wherein the melting point of the one or more hydrated vegetable fats is from 35° C. to 55° C.;
   10-30 wt.-% of one or more carbohydrates and/or sugar alcohols; and
   40-70 wt.-% of one or more solvents;
b) heating the formulation to a temperature wherein the formulation is at liquid state;
c) admixing the formulation at the liquid state with one or more active pharmaceutical ingredients (APIs) to produce a dispersion;
d) dispensing the dispersion to a substrate; and
e) cooling the dispersion, thereby providing a solid dosage formulation.

For providing a dispenseable dispersion, the total wt.-% of the one or more APIs in the dispersion is 0.1-25 wt.-% for APIs of Class I and Class III. When one or more of the APIs is of Class II or Class IV, the total wt.-% of the one or more APIs in the dispersion is 0.1-5 wt.-% for APIs of Class II and Class IV.

In an embodiment, the heating in step b) is to 35-55° C., which confirms that the formulation is in a liquid state. In an embodiment, this temperature is maintained during the admixing and the dispensing steps c) and d). In an embodiment, the cooling of step e) is to 25° C. or below.

According to a particular embodiment, one of more APIs are premixed with one or more surfactants, such as polysorbates, prior to admixing with the formulation. The use of a surfactant, such as Polysorbate 80, assists in the removal of dose formulations from substrates, in particular when blisters made of plastic are used as substrates.

The formulations of the present disclosure can also be used for dispensing APIs derived from crushed tablets, provided that the tablets include at least 50 wt.-% API.

In another aspect, the present disclosure also concerns a method for producing a solid dosage formulation, the method comprising the steps of:
a) providing a formulation comprising:
   5-30 wt.-% of one or more gelling agents selected from a group consisting of gelatin, agar, and combinations thereof;
   0.5-5 wt.-% silica;
   10-35 wt.-% of one or more lipid-based excipients selected from one or more hydrated vegetable fats, wherein the melting point of the one or more hydrated vegetable fats is from 35° C. to 55° C.;
   10-30 wt.-% of one or more carbohydrates and/or sugar alcohols; and
   40-70 wt.-% of one or more solvents;
b) admixing the formulation with:
   i. crushed tablets comprising at least 50 wt.-% of one or more active pharmaceutical ingredients, and
   ii. optionally one or more surfactants to produce an admixture; wherein
      the total wt.-% of the one or more APIs in the admixture is 0.1-25 wt.-% for APIs of Class I and Class III, and
      the total wt.-% of the one or more APIs in the admixture is 0.1-5 wt.-% for APIs of Class II and Class IV;
c) heating the admixture to a temperature wherein the formulation is at a liquid state to produce a dispersion;
d) dispensing the dispersion to a substrate; and
e) cooling the dispersion, thereby providing a solid dosage formulation.

In an embodiment, the heating of step c) is to 35-55° C. which confirms that the formulation is at liquid state. In an embodiment, the dispensing of step c) is done at 35-55° C. In an embodiment, the cooling of step e) is to 25° C. or below.

If the tablets originally included a coating, the coating may be removed from the crushed tablets, e.g., by sieving, prior to admixing with a formulation of the present disclosure.

According to a particular embodiment, the crushed tablets comprising one of more APIs are premixed with one or more surfactants, such as polysorbates, prior to admixing with the formulation. The use of a surfactant, such as Polysorbate 80, assists in the removal of the formulations from substrates, in particular when blisters are used as substrates.

Exemplary non-limiting APIs suitable for the present disclosure are those disclosed in Table 2.

When an API is added to the formulation, its effect on the viscosity will depend on which phase it will go into, i.e., lipophilic drugs into a lipid phase and hydrophilic drugs in a water/hydrophilic phase. Also, the API may decrease or increase the viscosity. The effects are typically governed by the solubility of the drugs in the matrix and hydrogen bonding. Substances that are capable of forming hydrogen bonds tend to have a higher viscosity than those that do not form hydrogen bonds. Generally, substances that have the possibility of multiple hydrogen bonds exhibit even higher viscosities. The hydrogen bonding potential is higher with higher concentrations and vice versa.

In some embodiments, the dispersion is heated to 35° C. or higher to ensure proper dispensing. In an embodiment, the dispensing temperature is between 37° C. and 55° C. An exemplary dispensing temperature is 40-42° C. Another exemplary dispensing temperature is 45° C.

The dispersion is dispensed onto a substrate which is preferably positioned inside a dispenser. When the dispensing is completed, the dispersion is allowed to solidify upon cooling, e.g. by decreasing temperature to 30° C. or below.

### Experimental

### Materials

Clopidogrel and Propranolol hydrochloride (APIs; both EP grade) were obtained from Dr. Reddy's Laboratories Ltd (India). Topiramate (from Topimax® 200 mg tablets) was obtained from Janssen-Cilag Oy. Solvents and chemicals used were of reagent grade.

Mini Medi-Cap® Plus™ Blisters (MD425, from MediDose Group, City, USA) with 3/16″ size was used as the dispensing substrate.

### Preparation of Dispenseable Formulations

### Method A

The 1 wt.-% API formulation consisted of 94.6 wt.-% of the formulation of Table 1, 2 wt.-% polysorbate 80 (PS 80) as a surfactant, 2.4 wt.-% citric acid for pH regulation, and 1 wt.-% the active pharmaceutical ingredient (API).

Similarly, the 2 wt.-% API formulation included 94.8 wt.-% of the formulation of Table 1, 2 wt-. % PS 80, 1.2 wt.-% citric acid for pH regulation, and 2 wt.-% API.

The process involved melting the formulation of Table 1 in a water bath at +45° C. (within a temperature range of ±3° C.) for 30 minutes until it transitioned from a solid to a liquid state. Clopidogrel and PS80 were measured in a metal mortar and mixed until forming a white paste. In incremental steps, warm formulation of Table 1 was introduced to a mortar while mixing. As the final step for pH adjustment, citric acid was incorporated. The formulation was thoroughly mixed for 3-4 minutes. Subsequently, the covered mortar was immersed in a warm water bath for 5 minutes and mixed once again to ensure consistent distribution of the API in the formulation.

The freshly prepared formulation was left undisturbed and securely covered in a water bath (42-45° C.) for 10-15 minutes before dispensing to eliminate air bubbles generated during mixing, thereby ensuring a successful deposition outcome.

The warm formulation was then transferred to a disposable sterile syringe (100 mL), expelling excess air until the first drops emerged from the nozzle. After capping the syringe, it was placed in the dispenser's holder and left to stand for 15 minutes at +41° C. before commencing the extrusion process.

### Method B

Topimax 200 mg tablets (31 tablets) were crushed using a mortar and a pestle. Film coating was removed by sieving the crushed mass using a 500 μm sieve.

Desired amount of sieved and crushed tables were mixed with Polysorbate 80 and the formulation of Table 1.

| Ingredient | wt.-% | wt.-% |
|---|---|---|
| Crushed and sieved Topamax 200 mg tablets | 14.29 | 7.16 |
| Formulation of Table 1 | 80.71 | 87.84 |
| Polysorbate 80 | 5.00 | 5.00 |
| Total | 100 | 100 |
| Topimarate in the admixture | 8.0 | 4.0 |

The admixtures were warmed to (42-45° C.) for 10-15 minutes. The warm dispersion was then transferred to a disposable sterile syringe (100 mL), expelling excess air until the first drops emerged from the nozzle. After capping the syringe, it was placed in the dispenser's holder and left to stand for 15 minutes at +41° C. before commencing the extrusion process.

### Deposition by Semi Solid Extrusion

The tablet production process utilized extrusion-based dosing equipment, e.g., from Pharma Printer (CurifyLabs Oy, Helsinki, Finland), which features a dispensing head specifically designed for compatibility with semi-solid extrusion.

The integration of the Pharma Printer involved linking it with an analytical balance (Kern PES-620-3M, Germany).

To meet diverse dosing needs, tablet sizes were varied, resulting in different strengths of the formulation. For example, 1, 2, 3, 4, and 5 mg of clopidogrel per tablet for the 1% formulation, and 2, 4, 6, 8, and 10 mg of clopidogrel per tablet for the clopidogrel 2% formulation were provided, thereby providing a wide range of therapeutic options.

### Drug Content and Dose Accuracy by HPLC

The data presented in Table 3 represents the content uniformity of the produced oromucosal tablets with varying concentrations of clopidogrel (1% and 2%) across four different tablet sizes (500 mg, 400 mg, 300 mg, and 200 mg). The evaluation involved key statistical parameters, such as the minimum, maximum, mean and AV, which is a critical indicator for content uniformity. The acceptance criteria for content uniformity were set at 85.0% to 115.0% with an AV less than 15, ensuring the reliability and consistency of the pharmaceutical product.

TABLE 3
Comparative analysis of content uniformity: 1 wt.-% and
2 wt.-% Clopidogrel formulations at various strengths.

| Dose | N | Min (%) | Max (%) | Mean (%) | AV^{a} |
|---|---|---|---|---|---|
| 1 wt-%; 200 mg | 10 | 99.3 | 104.1 | 102.0 | 5 |
| 1 wt-%; 300 mg | 10 | 96.7 | 104.8 | 99.7 | 4 |
| 1 wt-%; 400 mg | 10 | 97.5 | 104.8 | 101.7 | 5 |
| 1 wt.-%; 500 mg | 10 | 89.5 | 98.7 | 96.6 | 8 |
| 2 wt.-%; 200 mg | 10 | 92.3 | 106.0 | 102.6 | 10 |
| 2 wt.-%; 300 mg | 10 | 97.6 | 106.0 | 103.7 | 9 |
| 2 wt.-%; 400 mg | 10 | 89.5 | 105.8 | 101.5 | 12 |
| 2 wt.-%; 500 mg | 10 | 98.1 | 103.7 | 101.2 | 3 |

^{a}AV = Acceptance Value

Across all concentrations and tablet sizes, the mean values consistently fell within the acceptable range, indicating a reliable and precise manufacturing process. For instance, the mean clopidogrel content for 1% clopidogrel 500 mg tablets was 96.6%, well within the specified range.

Examining the AV for each tablet size and concentration, all values remained below the threshold of 15, underlining the adherence of the tablets to content uniformity standards. Notably, 2 wt.-% clopidogrel 500 mg exhibited the lowest AV of 3, indicating exceptional uniformity and reliability in the tablet's clopidogrel content.

The minimum and maximum values further supported the consistency observed in the mean and AV values. The narrow range between the minimum and maximum values for each tablet size and concentration showed consistent clopidogrel content, supporting the reliability of the manufacturing process across different tablet sizes. Table 4 shows the precision in dosing across different tablet sizes of formulations, including 1 wt.-% Propranolol as the API. Dosing weight increments of 25 mg were introduced. The testing protocol with 3×16 tablets for 200, 300, 400 and 500 mg targets weight was tested, and dosing was also demonstrated with target weights of 225, 275 and 425 mg respectively, which are three randomly chosen the targets weights in between the fixed values in the protocol. All in all, 336 tablets were dosed in this phase. The mean values for mass variation (MV) demonstrate consistent accuracy, with minimal deviations observed for all. The accuracy, as defined per Ph. Eur. Limits, for the tablets in all target weight classes was 100%. For the 225 mg tablets, the mass variation ranged from 218.0 to 230.0, with a mean of 224.5 and a standard deviation of 2.2. Similarly, for the 275 mg tablets, the mass variation remained tightly clustered, ranging from 264.0 to 281.0, with a mean of 274.0 mg and a standard deviation of 3.1. The 425 mg tablets also exhibited precision in dosing, with values concentrated around the mean of 423.6 and a standard deviation of 4.3.

TABLE 4

| tablet | N | min (mg) | max (mg) | mean (mg) | std. dev. |
|---|---|---|---|---|---|
| 200 mg | 48 | 193 | 208 | 200.5 | 3.2 |
| 300 mg | 48 | 289 | 314 | 300.8 | 5.0 |
| 400 mg | 48 | 382 | 420 | 401.8 | 6.4 |
| 500 mg | 48 | 485 | 521 | 502.0 | 6.7 |
| 225 mg | 48 | 218.0 | 230.0 | 224.5 | 2.2 |
| 275 mg | 48 | 264.0 | 281.0 | 274.0 | 3.1 |
| 425 mg | 48 | 408.0 | 437.0 | 423.6 | 4.4 |

### Conclusions

The formulations produced homogenous dispersions with the APIs tested.
The material stream out from the dispenser was uniform without undesired dripping, leading to seamless deposition of the formulation. Also, there was practically no extrusion of the formulation into the waste bin before the start of each dispensing job.
The formulations are homogenous with no separation up to 9-12 months at room temperature.
The mechanical properties for the formulations are excellent, thereby making handling easy.
The doses were not prone to stick on blister packages.
The dose from dose mass variation was low.
The physical appearance and mouthfeel was good.
The formulations kept their physical integrity and mechanical properties for long periods, even when exposed to open air.

It is contemplated that, unless stated otherwise, any of the embodiments and/or features of the embodiments described herein may be combined to form additional embodiments. Such combinations are understood within the scope of this description.

Further, it is understood that embodiments of the disclosure described above do not limit the scope of the invention, since these embodiments are merely examples of the embodiments of the invention, which are defined by the appended claims and their legal equivalents. Any equivalent embodiments are intended to be within the scope of this invention. Still further, various modifications of the disclosure, in addition to those described, such as alternative useful combinations of the embodiments described, may become apparent to those skilled in the art from the description. Such modifications and embodiments are also intended to fall within the scope of the appended claims.

What is claimed is:

1. A formulation for active pharmaceutical ingredients, the formulation comprising:
5-30 wt.-% of one or more gelling agents selected from a group consisting of gelatin, agar, and combinations thereof;
0.5-5 wt.-% silica;
10-35 wt.-% of one or more hydrated vegetable fats, wherein the melting point of the one or more hydrated vegetable fats is from 35° C. to 55° C.;
10-30 wt.-% of one or more carbohydrates and/or sugar alcohols; and
40-70 wt.-% of one or more solvents.

2. The formulation according to claim 1, wherein the one or more hydrated vegetable fats are selected from a group consisting of cocoa butter, hydrated coconut oil, and combinations thereof.

3. The formulation according to claim 1, wherein the one or more gelling agents comprise gelatin.

4. The formulation according to claim 1, wherein the formulation comprises one or more sugar alcohols, wherein the one or more sugar alcohols comprise xylitol, and wherein the formulation includes 10-30 wt.-% xylitol.

5. The formulation according to claim 1, wherein the one or more solvents are selected from a group consisting of water, glycerol, and combinations thereof.

6. The formulation according to claim 1, further comprising one or more preservatives, pH adjusting agents, flavoring agents, or sweeteners.

7. The formulation according to claim 1, further comprising citric acid, sodium citrate, or potassium sorbate.

8. The formulation according to claim 1, wherein the formulation comprises:
10-15 wt.-% gelatin;
0.5-5 wt.-% silica;
10-15 wt.-% cocoa butter;
40-45 wt.-% water;
5-6 wt.-% glycerol; and
15-25 wt.-% xylitol.

9. A dispersion comprising 95-99.1 wt.-% of the formulation of claim 1 and 0.1-5 wt.-% of one or more active pharmaceutical ingredients (APIs).

10. The dispersion according to claim 9, wherein the one or more APIs are selected from a group consisting of Amlodipine, Acetylsalicylic acid, Bisoprolol, Candesartan, Atenolol, Caffeine, Captopril, Clopidogrel, Diltiazem, Furosemide, Hydrochlorothiazide, Ibuprofen, Melatonin, Metoprolol, Ondansetron dihydrate, Paracetamol, Prednisolone, Propranolol, Quetiapine, Ramipril, Sildenafil, Spironolactone, Atenolol, Diltiazem, Gabapentin, Sotalol, Tacrolimus, Topiramate, Doxycycline, Ursodeoxycholic acid, Neomycin, Isoniazid, Nitrofurantoin, and pharmaceutically acceptable salts thereof.

11. A dispersion comprising 75-99.1 wt.-% of the formulation of claim 1 and 0.1-25 wt.-% of one or more active pharmaceutical ingredients of Class I and/or Class IV.

12. A method for producing a solid dosage formulation, the method comprising the steps of:
a) providing the formulation of claim 1;
b) heating the formulation to a temperature, wherein the formulation is at a liquid state;
c) admixing the formulation at the liquid state with one or more active pharmaceutical ingredients (APIs) to produce a dispersion,
wherein:
the total wt.-% of the one or more APIs in the dispersion is 0.1-25 wt.-% for APIs of Class I and Class III, and
the total wt.-% of the one or more APIs in the dispersion is 0.1-5 wt.-% for APIs of Class II and Class IV;
d) dispensing the dispersion to a substrate; and
e) cooling the dispersion, thereby providing a solid dosage formulation.

13. The method according to claim 12, wherein:
the heating of step b) is done to 35-55° C.;
the admixing of step c) is done at 35-55° C.;
the dispensing of step d) is done at 35-55° C.; and
the cooling of step e) is done to 25° C. or below.

14. The method according to claim 12, wherein step c) includes mixing the one or more active pharmaceutical ingredients (API) with a surfactant prior to the admixing.

15. The method according to claim 12, wherein the one or more APIs are selected from a group consisting of Amlodipine, Acetylsalicylic acid, Bisoprolol, Candesartan, Atenolol, Caffeine, Captopril, Clopidogrel, Diltiazem, Furosemide, Hydrochlorothiazide, Hydrocortisone, Ibuprofen, Melatonin, Metoprolol, Ondansetron dihydrate, Paracetamol, Prednisolone, Propranolol, Quetiapine, Ramipril, Sildenafil, Spironolactone, Atenolol, Diltiazem, Gabapentin, Sotalol, Tacrolimus, Topiramate, Doxycycline, Ursodeoxycholic acid, Neomycin, Isoniazid, Nitrofurantoin, and pharmaceutically acceptable salts thereof.

16. The method according to claim 12, wherein the substrate is a blister.

17. A method for producing a solid dosage formulation, the method comprising the steps of:
a) providing the formulation of claim 1;
b) admixing the formulation with:
i. crushed tablets comprising at least 50 wt.-% of one or more active pharmaceutical ingredients (APIs), and
ii. optionally one or more surfactants to produce an admixture, wherein
the total wt.-% of the one or more APIs in the admixture is 0.1-25 wt.-% for APIs of Class I and Class III, and
the total wt.-% of the one or more APIs in the admixture is 0.1-5 wt.-% for APIs of Class II and Class IV;
c) heating the admixture to a temperature, wherein the formulation is at liquid state to produce a dispersion;
d) dispensing the dispersion to a substrate; and
e) cooling the dispersion, thereby providing a solid dosage formulation.

18. The method according to claim 17, wherein:
the heating of step c) is done to 35-55° C.;
the dispensing of step d) is done at 35-55° C.; and
the cooling of step e) is done to 25° C. or below.

19. The method according to claim 17, wherein step b) includes mixing the crushed tablets with a surfactant prior to the admixing.

20. The method according to claim 17, wherein the one or more APIs are selected from a group consisting of Amlodipine, Acetylsalicylic acid, Bisoprolol, Candesartan, Atenolol, Caffeine, Captopril, Clopidogrel, Diltiazem, Furosemide, Hydrochlorothiazide, Hydrocortisone, Ibuprofen, Melatonin, Metoprolol, Ondansetron dihydrate, Paracetamol, Prednisolone, Propranolol, Quetiapine, Ramipril, Sildenafil, Spironolactone, Atenolol, Diltiazem, Gabapentin, Sotalol, Tacrolimus, Topiramate, Doxycycline, Ursodeoxycholic acid, Neomycin, Isoniazid, Nitrofurantoin, and pharmaceutically acceptable salts thereof.

21. (canceled)
